(19) United States
(12) Patent Application Publication
(10) Pub. No.: US 2007/0248680 A1
(43) Pub. Date: Oct. 25, 2007

(54) Particles for Inactivating Toxins

(75) Inventor: Liping Tang, Arlington, TX (US)

Correspondence Address: GARDERE WYNNE SEWELL LLP;INTELLECTUAL PROPERTY SECTION, 3000 THANKSGIVING TOWER, 1601 ELM ST, DALLAS, TX, 75201-4761, US

(73) Assignee: Board of Regents, The University of Texas System, Austin, TX (US)

(21) Appl. No.: 11/660,282

(22) PCT Filed: Feb. 8, 2006
(86) PCT No.: PCT/US06/04361

§ 371 Date: Feb. 15, 2007

Related U.S. Application Data
(60) Provisional application No. 60/650,805, filed on Feb. 8, 2005.

(51) Int. Cl. A61K 9/14; A61P 31/00

(52) U.S. Cl. 424/490; 424/489

(57) ABSTRACT

The present invention provides for compositions and methods for recognizing toxins in a host. The composition comprising a particle of less than 100 micrometers in diameter having a tag in direct contact with a portion of the particle, the particle comprising a biodegradable polymer and the portion of the particle in contact with the tag selected from the group consisting of acrylic acid, 2-hydroxyethyl acrylate, 2-acrylamido-2-methyl-1-propanesulfonic acid, allylamine, carboxyl group, hydroxyl group, sulfonic group, aldehyde group, and amine group, wherein the tag is selected from the group consisting of drug, antibody, ligand, antigen, protein, peptide, counter-ligand moiety, fatty acid moiety, and carbohydrate moiety, and wherein one or more particles are introduced into the host having toxins and the one or more particle recognize the toxins.

### CROSS REFERENCE TO RELATED APPLICATION

This application claims the benefit of U.S. Provisional Patent Application No. 60/650,805, filed Feb. 8, 2005.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of EB-00287 awarded by The National Institutes of Health.

### BACKGROUND OF THE INVENTION

The present invention relates generally to the field of microbiology and in particular to recognizing and inactivating and/or eliminating toxins (e.g., antibodies, immune products, host cell by-products or metabolites, chemicals, and/or microorganisms and products produced therefrom) in a host through the introduction of microscopic particles (i.e., microparticles) or submicroscopic particles (e.g., nanoparticles).

Many diseases are launched by the direct contact and accumulation of toxic agents on a host (i.e., its cells and tissues). These diseases include autoimmune diseases, complement component-mediated diseases and microorganism-induced (e.g., infectious) diseases. Removal of such toxic agents typically relies on drug treatments to eradicate the agent. Most drug treatments, however, are not effective in all hosts and are often less effective or become ineffective with more widespread use. Antimicrobials, for example, are used extensively in the treatment of infectious diseases. Yet, the majority of antimicrobials are truly effective in only specific segments of the population.

Other methods designed to remove toxic agents from a host include cell ligand-masking techniques and the use of inactivating agents. While ligand-masking techniques successfully diminish the infectious properties of a microorganism in vitro, the techniques have generally failed to show therapeutic efficacy in vivo. Similarly, inactivating agents work in vitro, but not in vivo. The largest problem with such techniques is that the inactivating agents or counter-ligands (e.g., protein, peptide, microorganism-inhibitor, etc.) that are available typically exhibit very short half lives in vivo and are, thus, ineffective at inactivating the agent or microorganism or removal thereof. As such, there remains a need to effectively recognize toxins in a host (e.g., microorganisms and products (e.g., antibodies, immune products, host cell by-products or metabolites, and/or microorganisms and products produced therefrom) in order to inactivate and/or eradicate the toxin or its product from the host.

### SUMMARY OF THE INVENTION

The present invention solves many problems associated with the introduction of a toxin or toxic agent into a host.

Generally, and in one form the present invention provides for a particle preparation comprising a particle in contact with one or more tags that specifically recognize a toxin (e.g., recognition molecule, surface molecule, cell-surface receptor, antigen, ligand-masking moiety). The particle is typically prepared using biocompatible or degradable polymers. When administered to a host in need thereof, the particle preparation recognizes the toxin in the host and inactivates, removes and/or reduces the number (or concentration) of such toxins in the host. The particle preparation is typically administered by routes known to one or ordinary skill, including by injection, infusion, inhalation, mouth, transdermally, suppository, by drops or as a lubricant.

In another form, the present invention is a particle preparation that recognizes auto-antibodies and is capable of depleting the auto-antibodies from circulation when introduced into a host in need thereof.

In yet another form, the present invention is a particle preparation that recognizes unwanted microorganisms and is capable of contacting the microorganism, preventing their accumulation and or removing such unwanted microorganisms from a host when introduced into the host in need thereof.

Advantages of compositions of the present invention when administered into a host in need thereof include the ability of such compositions to: (a) effectively remove one or more toxins or their unwanted products from the host, such as from the circulation, often in a very short period of time; (b) being effective in a host for long periods of time, longer than other products/materials used to remove toxins from a host; (c) recognize one or more toxins or their unwanted products and prevent further deleterious actions; (d) neutralize toxins or their unwanted products without the need for an additional drug therapy. In addition, compositions of the present invention do not appear to provide negative side effects in the host that they are introduced into and do not appear to alter normal immune responses in the host.

This invention may be preventative or curative when introduced to a host in need thereof, especially with regard to diseases initiated by the accumulation of one or more toxins. There present invention may be used in vitro, in vivo or ex vivo, as needed.

Those skilled in the art will further appreciate the above-noted features and advantages of the invention together with other important aspects thereof upon reading the detailed description that follows in conjunction with the drawings.

### BRIEF DESCRIPTION OF SEVERAL VIEWS OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 depicts a schematic of a particle in accordance with one aspect of the present invention;

FIG. 2 depicts a schematic of a particle in accordance with another aspect of the present invention;

FIG. 3 depicts a schematic of tagged particle activity in accordance with one aspect of the present invention;

FIG. 4 depicts a schematic of tagged particle activity in accordance with another aspect of the present invention;

FIGS. 5A and 5B show the lasting effect of particles in the blood in accordance with one aspect of the present invention;

FIG. 6 depicts an analysis of tagged particle recognition in accordance with one aspect of the present invention;

FIG. 7 depicts an analysis of tagged particle activity in accordance with one aspect of the present invention;

FIGS. 8A and 8B depict tagged particle activity in accordance with yet another aspect of the present invention;

FIGS. 9A and 9B depict tagged particle specificity in accordance with one aspect of the present invention

FIGS. 10A and 10B depict bacterial survival analysis with tagged and untagged particles in accordance with one aspect of the present invention;

FIGS. 11A and 11B depict CD4+ and CD14+ cell analyses, respectively, with tagged and untagged particles in accordance with another aspect of the present invention; and

FIG. 12 depicts the extended half-life of molecules after association with particles of the present invention.

### DETAILED DESCRIPTION

Although making and using various embodiments of the present invention are discussed in detail below, it should be appreciated that the present invention provides many inventive concepts that may be embodied in a wide variety of contexts. The specific aspects and embodiments discussed herein are merely illustrative of ways to make and use the invention, and do not limit the scope of the invention.

In the description which follows, like parts may be marked throughout the specification and drawing with the same reference numerals, respectively. The drawing figures are not necessarily to scale and certain features may be shown exaggerated in scale or in somewhat generalized or schematic form in the interest of clarity and conciseness.

The present invention provides a method to produce biocompatible, degradable or non-degradable nano- and micro-particles capable of recognizing and removing toxins from a host presenting such toxins. The particles themselves are typically compatible with a host's blood. The host is typically a mammal. The toxins are unwanted molecules, molecular complexes or microorganisms (e.g., bacteria, virus, fungus) and their detrimental products or metabolites, such as autoimmune antibodies, bilirubin, and other detrimental products produced in response to an accumulation of toxins in the host, such as immune products, host cell by-products or metabolites.

Autoimmune diseases are triggered by the overproduction of auto-antibodies by host tissues/cells. Particles of the present invention are functionalized and tagged with one or more specific recognition molecules (e.g., autoimmune molecule or cell surface antigen) capable of recognizing one or more unwanted toxins introduced or produced with the disease. The tags (recognition molecules) may recognize antibodies (e.g., auto-antibodies overproduced by the host), antibody complexes (e.g., antigen-presenting cells, antibody-producing cells) or autoimmune by products. Administration of such particle preparations into a host in need thereof provides for recognition of the toxin by the particle preparation and removal of the toxin(s) from the host. Recognition results in host protection from further production and/or accumulation of the toxin.

Microorganism infection often includes the attachment of the microorganism to the surface of one or more cells. For example, a virus typically recognizes a receptor at the cell surface in order to invade the cell. Many cell receptors (e.g., cell adhesion molecules or CAMs) recognized by microorganisms are known. Particles of the present invention are functionalized and tagged with one or more specific recognition molecules capable of recognizing one or more unwanted microorganisms or toxins introduced or produced therefrom. The recognition molecule may include CAMs. When introduced to a host in need, particle preparations recognize the unwanted microorganism or toxins introduced or produced therefrom and prevent of the microorganism or toxins from further accumulation. In addition, particle preparations now in contact with the microorganism or toxin diminish colonization and promote removal of the microorganism or toxin from the host without the need for additional therapy, such as antimicrobial therapy and/or vaccination.

The above inventive aspects of the present invention do not depend on a particular particle composition, as long as the particle is tagged with a specific toxin-recognizing molecule. As used herein, the particle preparation includes particles which are particle-like structures that are microscopic or submicroscopic (e.g., microparticles, nanoparticles) with portions of the particle capable of recognizing one or more toxins. Particle preparations typically comprise a polymer and a tag which is a toxin-recognizing molecule, compound or complex. Additional components may include one or more metals, proteins, coating agents, carriers, nucleic acids, microorganisms and combinations thereof. The particle preparation may have additional surface modifications to enhance specificity or recognition. The surface of the particle preparation has one or more tags used for recognizing a toxin, which may also be used for labeling and/or locating the particle preparation upon introduction into a host in need thereof.

Particles of the present invention are a few nanometers in size up to few millimeters in size, often submicroscopic (less than one micrometer) and typically have an average diameter of less than 100 micrometers. The particles are solid colloidal objects that may be cylindrical or spherical in shape with a semipermeable shell or shaped like a permeable nano-ball.

The tag is a toxin-recognizing molecule, compound or complex and may be used for diagnosis or therapy. The tag may also include a label or locator, such as a light or color absorbing dye, isotope, radioactive species, and/or organic or inorganic molecule. Inclusion of one or more tags to the surface of a particle occurs by entrapment, encapsulation, absorption, adsorption, covalent linkage, or other contact process known to one of ordinary skill in the art. When a tag is in contact with a particle of the present invention, it may be adsorbed or absorbed to a pre-made particle, or incorporated into the particle during the manufacturing process. Methods of absorption, adsorption, and incorporation are of common knowledge to those skilled in the art. The choice of the particle preparation (e.g., polymer, solvent, emulsifier, tag and other auxiliary substances used herein) are typically dictated by the toxin and the host in need thereof. Fabricating the particle preparation may be performed without limitation and difficulty by those skilled in the art. The ratio of tag to particle may be varied as required.

As used herein, a tag has an ability to modify the particle. Tags may include drugs and/or molecular ligands (e.g., molecules/compounds) that recognize a portion of the toxin. Examples of a tag are an antibody, antigen, protein, peptide, counter-ligand, growth factor, nucleic acid sequence, fatty acid, carbohydrate moiety, and chemical. A tag may also be a modified compound or polymer that mimics the site for recognition on the toxin. The site for recognition on a toxin may include, but is not limited to, a cell surface marker, cell surface receptor, immune complex, antibody, MHC, extracellular matrix protein, cell membrane, protein, polypeptide, cofactor, growth factor, fatty acid, lipid, carbohydrate chain, cytokine, as examples.

Particles of the present invention typically comprise one of the materials selected from the following: biodegradable polymer, nonbiodegradable polymer, metal, magnetic material, inorganic chemical, organic chemical, ceramic, graphite, and may be a hydrogel particle, in liquid form, and/or porous (with or without gas-filled pores).

Suitable polymers of the present invention include copolymers of water soluble polymers, including, but not limited to, dextran, derivatives of poly-methacrylamide, PEG, maleic acid, malic acid, and maleic acid anhydride and may include these polymers and a suitable coupling agent, including 1-ethyl-3 (3-dimethylaminopropyl)-carbodimide, also referred to as carbodimide. Polymers may be degradable or nondegradable or of a polyelectrolyte material. Degradable polymer materials include poly-L-glycolic acid (PLGA), poly-DL-glycolic, poly-L-lactic acid (PLLA), PLLA-PLGA copolymers, poly(DL-lactide)-block-methoxy polyethylene glycol, polycaprolacton, poly(caprolacton)-block-methoxy polyethylene glycol (PCL-MePEG), poly(DL-lactide-co-caprolactone)-block-methoxy polyethylene glycol (PDLLACL-MePEG), some polysaccharide (e.g., hyaluronic acid, polyglycan, chitoson), proteins (e.g., fibrinogen, albumin, collagen, extracellular matrix), peptides (e.g., RGD, polyhistidine), nucleic acids (e.g., RNA, DNA, single or double stranded), viruses, bacteria, cells and cell fragments, organic or carbon-containing materials, as examples. Nondegradable materials include natural or synthetic polymeric materials (e.g., polystyrene, polypropylene, polyethylene teraphthalate, polyether urethane, polyvinyl chloride, silica, polydimethyl siloxane, acrylates, arcylamides, poly (vinylpyridine), polyacroleine, polyglutaraldehyde), some polysaccharides (e.g., hydroxypropyl cellulose, cellulose derivatives, dextran®, dextrose, sucrose, ficoll®, percoll®, arabinogalactan, starch), and hydrogels (e.g., polyethylene glycol, ethylene vinyl acetate, N-isopropylacrylamide, polyamine, polyethyleneimine, poly-aluminum chloride).

Particles of the present invention may be coated prior to tagging, as required. The coating, tag or both may be added for specificity or to further increase affinity. Should particles of the present invention require an additional layer or coating, suitable coatings include, as examples, surfactants, light-emitting species, hydrophilic spacers, radioactive species, nuclear species, and combinations thereof. The surfactants include fatty acid esters of glycerols, sorbitol and other multifunctional alcohols (e.g., glycerol monostearate, sorbitan monolaurate, sorbitan monoleate), polysorbates, poloxamers, poloxamines, polyoxyethylene ethers and polyoxyethylene esters, ethoxylated triglycerides, ethoxylated phenols and ethoxylated diphenols, surfactants of the Genapol™ and Bauki series, metal salts of fatty acids, metal salts of fatty alcohol sulfates, sodium lauryl sulfate, and metal salts of sulfosuccinates.

Particle preparations of the present invention may be provided to a host in need thereof, the host having one or more toxins. One or more particles having a tag are typically introduced to the host by any of a number of routes of administration known to one of ordinary skill (e.g., infusion, injection, inhalation, by mouth, transdermally, by suppository, by drops, or lubricant). Typically, a portion of the particle is selected from the group consisting of acrylic acid, 2-hydroxyethyl acrylate, 2-acrylamido-2-methyl-1-propanesulfonic acid, allylamine, carboxyl group, hydroxyl group, sulfonic group, aldehyde group, and amine group. The average particle has a typical diameter of at least or less than 100 micrometers. Upon introduction to a host in need thereof, the particle preparation is, thus, protective or therapeutic to the host. Often, the host in need exhibits symptoms, such as an infection or a disease, as a result of having the toxin.

The particle preparations of the present invention are produced by conventional methods known to those of ordinary skill in the art. Techniques include emulsion polymerization in a continuous aqueous phase, emulsion polymerization in continuous organic phase, interfacial polymerization, solvent deposition, solvent evaporation, dissolvation of an organic polymer solution, cross-linking of water-soluble polymers in emulsion, dissolvation of macromolecules, and carbohydrate cross-linking. These fabrication methods can be performed with a wide range of polymer materials as described above. Removal of any solvent or emulsifier as required may include a number of methods well known to one of ordinary skill in the art. Examples of materials and fabrication methods for making particles have been published. (See Kreuter, J. 1991, Nanoparticles-preparation and applications; In: M. Donbrow (Ed.), Microcapsules and nanoparticles in medicine and pharmacy. CRC Press, Boca Raton, Fla., pp. 125-148; Hu, Z, Gao J. Optical properties of N-isopropylacrylamide microgel spheres in water. Langmuir 2002; 18:1306-67; Ghezzo E, et al., Hyaluronic acid derivative microspheres as NGF delivery devices: Preparation methods and in vitro release characterization. Int J Pharm 1992;87:21-29; all references incorporated herein by reference).

Example of a Particle Preparation: Synthesis of Hydroxypropyl Cellulose Particles

In one embodiment, hydroxypropyl cellulose (HPC) particles are synthesized by chemically crosslinking collapsed HPC polymer chains in salt water without any surfactant above the lower critical solution temperature (LCST) (at least about 41 degrees Centigrade). Methods include modifications from published method. (See Gehrke S H, Synthesis, Equilibrium Swelling, Kinetics Permeability and Applications of Environmentally Responsive Gels. Adv Polym Sci. 1993;110:81; Lu X H, Hu Z B, Gao J, Synthesis and Light Scattering Study of Hydroxypropyl Cellulose Microgels. Macromolecules. 2000;33:8698-702; all incorporated herein by reference). The size distributions of HPC particles may change by varying surfactant concentration, polymer concentrations, crosslinker densities, and reaction temperatures, as is known to one of ordinary skill in the art.

Example of Preparing a Particle: Synthesis of N-isopropylacrylamide Particles

N-isopropylacrylamide (NIPA) particles were synthesized following disclosed methods with specific modifications. Different building blocks of NIPA-derivative particles, with various particle sizes and crosslinker densities, are synthesized using an emulsion polymerization method. (See Pelton R H, Chibante P, Preparation of Aqueous Latices with N-Isopropylacrylamide. Colloids and Surfaces. 1986;20:247-56; incorporated herein by reference.)

Particle examples of the present invention include NIPA co-polymerized with acrylic acid (AA), NIPA with 2-hydroxyethyl acrylate (HEAc), NIPA with HEAc and 2-acrylamido-2-methyl-1-propanesulfonic acid (AAMPSA) and NIPA with allylamine. The NIPA has thermally responsive properties; the AA, the HEAc, the AAMPSA, and the allylamine provide aldehyde, carboxyl (—COOH), hydroxyl (—OH), sulfonic (—SO₃—), and amine (NH₃) groups, respectively, for binding biomolecules (e.g., molecular ligands), drugs or other tags.

Example of Preparing a Particle: Synthesis of Hyaluronan and Derivative Particles

Hyaluronan (HA) is a molecule with biologic origin and biodegradable properties. HA particles were synthesized using modified procedures. (See Ghezzo E, et al., Hyaluronan derivative microspheres as NGF delivery devices: Preparation methods and in vitro release characterization. Int J Pharm. 1992;87:21-9; incorporation by reference, herein.) For the present invention, an oil-water emulsion is prepared in the internal phase (as at least about 6% HA) and the external phase is a mineral oil containing different amounts of surfactant (e.g., Arlacel®). Following mixing and stirring, ethyl acetate, the extraction solvent, is added to the emulsion (at least about 2:1 v/v) to form HA particles.

Example of Preparing a Particle: Synthesis of Poly-L-Lactic Acid and Derivative Particles

Poly-L-lactic acid (PLLA) is a 173 kD protein. PLLA particles were synthesized using an emulsion process. In one example, 0.45 g of PLLA was dissolved in 3 mL methylene chloride to form a solution to which was added 0.3 mL of deionized water. The mixture was mixed (e.g., vortexed) for about 15 minutes to form a primary emulsion. A secondary emulsion was then formed by adding 6.0 mL 2% polyvinyl alchohol followed by rigorous mixing. The secondary emulsion was added to 150 mL of deionized water and stirred at room temperature to allow particle formation. Particles formed were typically 150 micrometers in diameter. After solvent evaporation, particles were washed repeatedly and ready for use. Specific size particles were obtained by methods known in the art.

Production of Particle Preparations

A series of HPC and HA particles with functional hydroxyl groups were oxidized (e.g., with pyridinium chlorochromate and then hydazide to form CONHNH2 group). When conjugated with an antibody, the hydroxyl groups of the F(ab)2 were oxidized with sodium periodate to form an aldehyde group. Hydrazide (on HPC and HA particles) and amine (on NIPA particles) were then reacted with the aldehyde group on the F(ab)2 to form a covalent bond. Tagged, antibody-conjugated particles were ready for use after dialysis with sterile saline and able to cross one or more physiologic barriers. Other tags included BSA, collagen, albumin, and CD4 or CD14 antibodies.

A series of NIPA particles with amine functional groups of different sizes (approximately 10 μm to 50 nm typical diameter) were produced and then conjugated with a tag (e.g., BSA, collagen, fibronectin, CD4 antibody, fluorescein-isothiocyanate [FITC]). These particle preparations were able to cross one or more physiologic barrier.

A series of PLLA particles with amine functional groups of different sizes (1-50 micrometers typical diameter) were produced and then conjugated with a tag (e.g., BSA, fibronectin, CD14 antibody, FITC) These functionalized and tagged particles were able to cross one or more physiologic barrier.

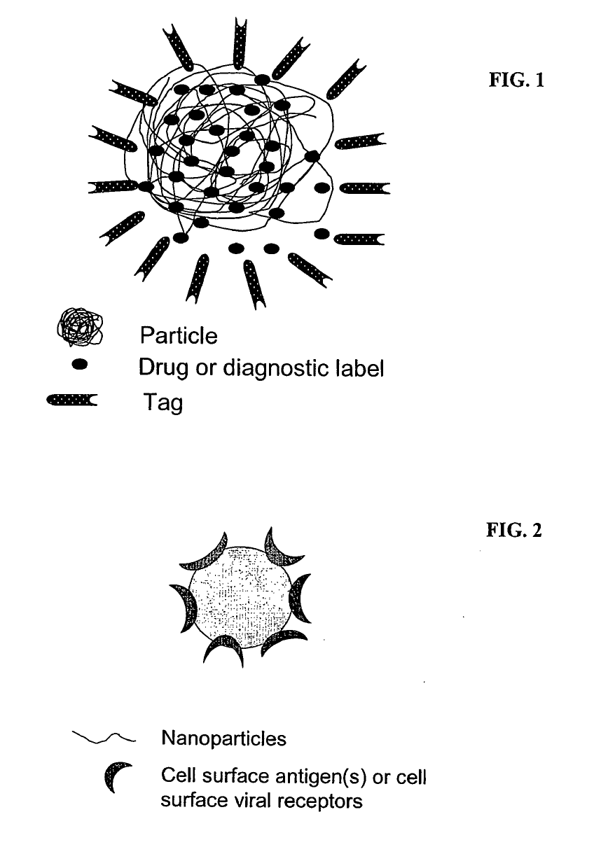

FIG. 1 is a schematic of a “smart” particle preparation of the present invention that is functionalized, tagged and now capable of inactivating a toxin. The particle is functionalized and has a tag. For treatment from invasion by a microorganism and/or prevention of microorganism accumulation, the tag is a recognition molecule (e.g., cell surface molecule, antibody, antigen, or other molecular compound or complex that contacts the particle, either by covalently binding to the surface, conjugation, or by blending it with the particle during particle formation). Often; the recognition molecule is provided as a covalent modification to the outer surfaces of the functionalized particle. To locate or further identify the smart particle there may be additional modifications to the particle, such as the addition of a drug or diagnostic label.

FIG. 2 is a schematic of a particle preparation of the present invention in which the particle preparation is a nanoparticle with a high affinity cell surface molecular tag (e.g., antigen [Ag] or virus-recognizing receptor) in contact with the surface of the nanoparticle.

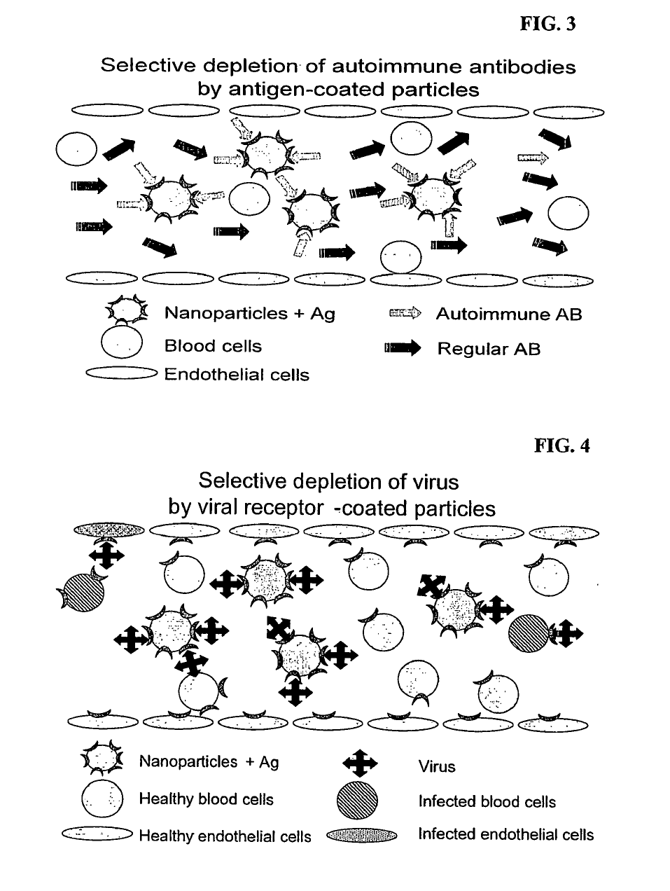

FIG. 3 is a schematic of one embodiment of the present invention in which particle preparations of FIG. 2 (nanoparticle+Ag) are introduced into a host circulation and found in a blood vessel and then recognize and collect autoimmune antibodies (AB, thinner arrows) circulating in the blood vessel. By collecting the autoimmune antibodies, particle preparations of the present invention prevent the autoimmune antibodies from causing further damage to the host.

FIG. 4 is a schematic of another embodiment of the present invention in which particle preparations of FIG. 2 (nanoparticle+Ag) are located in a blood vessel of a host and recognize and collect viruses (double arrows) circulating in the blood vessel. By collecting the viruses, the particle preparations of the present invention prevent further viruses from entering a cell and causing additional damage to the host.

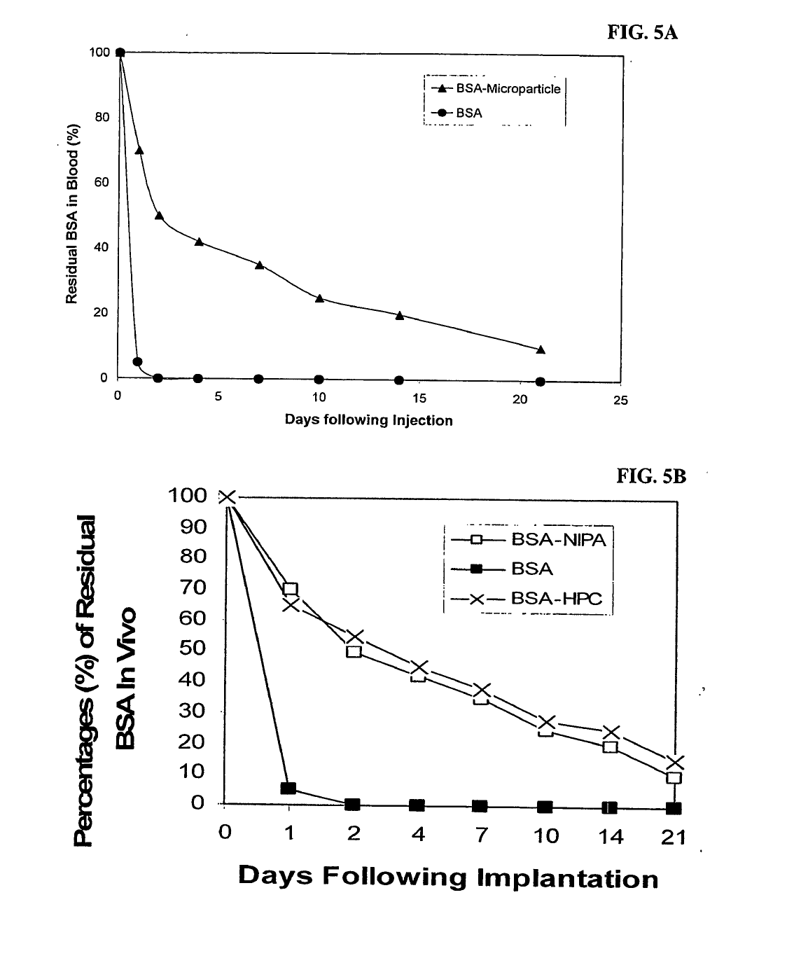

One feature of the present invention is that a particle preparation of the present invention is capable of remaining in the host for a length of time, longer than proteins, antibodies, vaccines, or antimicrobials are known to last in a mammalian host. FIGS. 5A and 5B show that particles as described herein extended the in vivo life-span of a tag (e.g., cell recognition molecule, antibody, antigen, other molecular ligand). Particle were tagged by conjugating with FITC-labeled bovine serum albumin (BSA) protein. BSA-tagged particles or BSA alone were introduced intravenously into two groups of Balb/C mice (at approximately 100 micrograms of BSA per 1 mg of particles; each group having at least 5 mice) which was followed by the collection of blood samples from each mouse at specific time points using retro-orbital blood collection methods. The amount of BSA present in each sample was assessed by fluorometry. The tagged particles in FIG. 5A were FITC-BSA conjugated to NIPA particles of about 5.0 micrometers (typical diameter). FIG. 5A further shows that BSA-NIPA particles (BSA microparticles) remained in the circulation longer than BSA alone (BSA); BSA alone was present in the blood less than two days while BSA-NIPA particles remained in the circulation for at least 2 weeks. In addition, greater than 20% of the BSA-NIPA particles were still present in the blood after two weeks.

In FIG. 5B, particles were HPC (approximately 5.0 micrometers, typical diameter) conjugated to BSA which were introduced intravenously into Balb/C mice and compared with mice injected with FITC-labeled BSA. As depicted in FIG. 5B, greater than 20% of BSA-HPC particles (BSA-HPC) were present in the host even after two weeks as compared to the virtual absence of labeled BSA (BSA) by day 2. FIG. 5B includes the BSA-NIPA particle data of FIG. 5A to show that behavior was similar for both preparations; both examples were prepared in the same manner and under the same conditions.

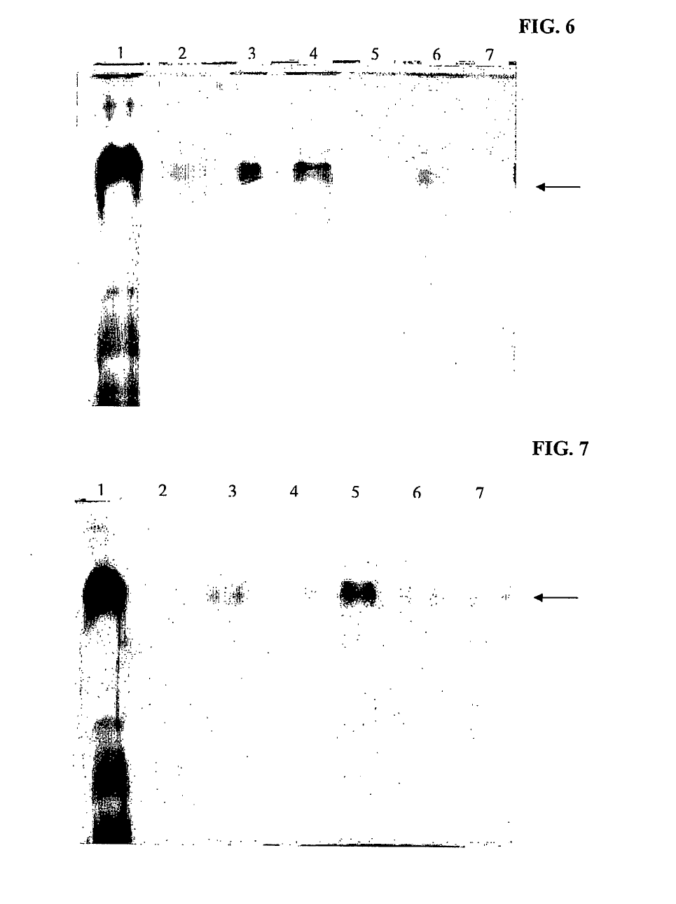

Another feature is that a tagged particle of the present invention may specifically recognize proteins or other host components of interest and as desired. For example, tagged particles were made to recover or eliminate BSA antibodies from immunized animals. First, Balb/C mice were immunized with bovine serum albumin (BSA) protein for four weeks to trigger BSA antibody production. Two weeks later, mice were sacrificed and their serum was recovered for analyses. The recovered serum was incubated with particles alone as NIPA microparticles (approximately 5 micrometers, typical diameter) alone or tagged particles of a similar size as NIPA microparticles conjugated with BSA. After incubation for at least about 2 hours, particles were recovered by centrifugation. From a portion of each sample of recovered particles, proteins were eluted. Particles and eluted proteins were analyzed by SDS-PAGE gel electrophoresis to identify proteins that had adsorbed to the particles. FIG. 6 is a representative gel showing tagged particle recognition of a specific antigen, which, in this case, is represented by bovine serum albumin.

In FIG. 6, lane 1 is diluted mouse sera in PBS at concentration of 1 mg/mL; lanes 2, 5 and 7 depict proteins eluted from NIPA microparticles preincubated with antisera from three different mice; lanes 3, 4 and 6 depict proteins eluted from NIPA-BSA microparticles preincubated with antisera from three different mice. Lanes 2 and 3 are samples from a first mouse, lanes 4 and 5 are samples from a second mouse, and lanes 6 and 7 are samples from a third mouse. FIG. 6 shows that NIPA microparticles alone adsorbed to or bound very few proteins, while BSA-NIPA microparticles recognized a number of proteins, especially those representing immunoglobulin G, as shown in lanes 3, 4, and 6. The predominant protein was found to recognize the antibody to BSA (see arrow).

In another example depicting the specific recognition of tagged particles, a similar immunization strategy was used. Here, tagged particle recognized only its target antibody and not other antibodies. First, Balb/C mice were immunized with bovine serum albumin (BSA) for 4 weeks to trigger BSA antibody production. Control mice were immunized with saline for the same time period. After 6 weeks, all mice were sacrificed and serum was recovered for analyses. Sera from BSA-immunized animals had BSA-specific antibodies. Such BSA-specific antibodies were not present in sera obtained from control animals. Sera samples collected from BSA-immunized animals were then incubated for two hours with either BSA-NIPA microparticles or unconjugated NIPA microparticles (particles as described above). After centrifugation, particles were recovered and proteins eluted from each sample. Eluted proteins were analyzed using SDS-PAGE gel electrophoresis. FIG. 7 is a representative gel showing tagged particle recognition and removal of a specific antigen.

In FIG. 7, lane 1 is BSA antisera in PBS at a concentration of 1 mg/mL; lane 2 shows proteins eluted from NIPA-BSA microparticles incubated with serum from a first mouse before immunization; lane 3 shows proteins eluted from NIPA-BSA microparticles incubated with serum from the first mouse after immunization; lane 4 shows proteins eluted from NIPA microparticles incubated with serum from the first mouse after immunization; lane 5 shows proteins eluted from NIPA-BSA microparticles incubated with BSA antisera from a second immunized mouse; lane 6 shows proteins eluted from NIPA-BSA microparticles incubated with serum from the second mouse before immunization; and lane 7 shows proteins eluted from NIPA microparticles incubated with BSA antisera from the first immunized mouse. FIG. 7 shows that NIPA particles tagged with BSA recognized more proteins (see lanes 3 and 5) from immunized serum than from non-immunized serum (lanes 2 and 6). Untagged (non-conjugated) NIPA microparticles recognized very few proteins (see lanes 4 and 7). The predominate protein eluted in this example was the antibody against BSA (see arrow).

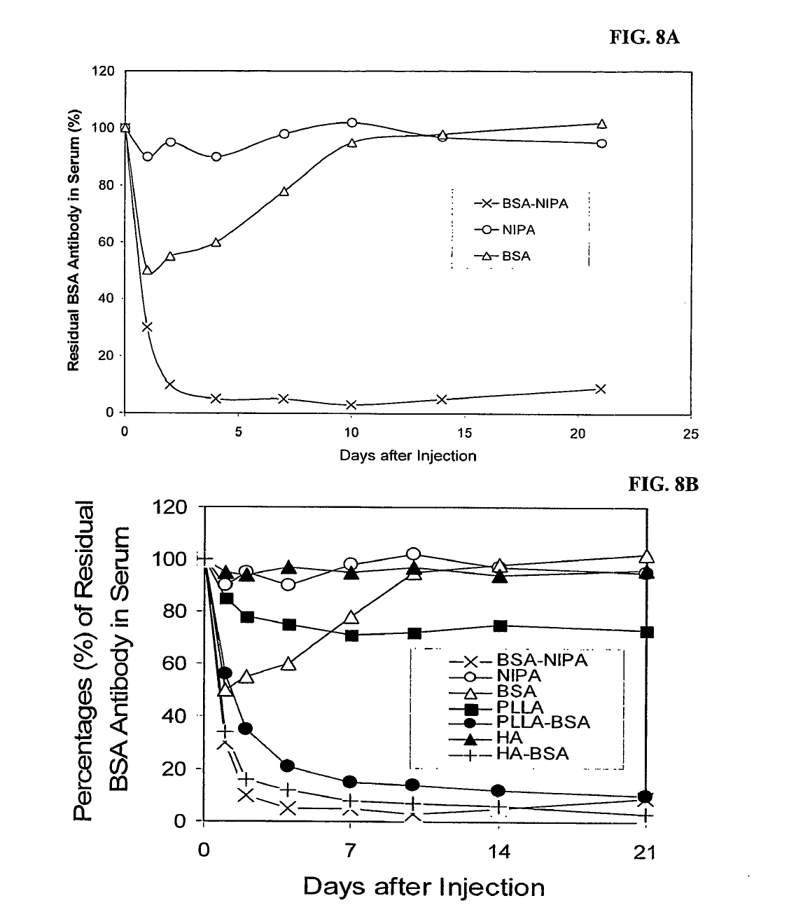

Another feature of the present invention is that selective tagging of particles of the present invention provides for the removal of select and specific unwanted products or toxins (e.g., unwanted proteins or antibodies) from a host. In the examples that follow, an immunization strategy was used in which Balb/C mice were immunized with BSA for 4 weeks to trigger BSA antibody production. At 6 weeks, mice were injected intravenously (via tail vein injection) with particles alone (untagged) or particles of a similar size tagged with BSA. Following injection, mice (at least five mice per group) were sacrificed beginning on day one and continued to day 21. Serum samples from each mouse were recovered via retro-orbital blood collection. Sera was then used to measure the amount of free BSA antibody in the blood by standard enzyme-linked immuno-sorbent assay. FIGS. 8A and 8B are representative examples of the efficacy of specifically tagged particles, in which tagged particles are capable of removing a specific antibody present in serum.

In FIG. 8A, mice were treated as described above with NIPA particles (approximately 5 micrometers, typical diameter) tagged with BSA, untagged NIPA particles of similar size or BSA alone. As depicted in the figure, mice that received tagged NIPA particles (BSA-NIPA) exhibited very low (<5%) free BSA antibodies in the serum as compared with those treated with BSA (BSA) or with untagged NIPA particles (NIPA), indicating that tagged particles recognized and removed most of the circulating BSA antibodies in the host. The removal of BSA antibodies with tagged BSA-NIPA particles was rapid and virtually complete by day 2. On the other hand, injection of either untagged NIPA particles or BSA alone did little to effect the amount of free and circulating BSA antibody detected in the host's serum.

In FIG. 8B, mice were similarly treated (as described above, under the same conditions) and injected in one example with PLLA microparticles (approximately 10 micrometer, typical diameter), untagged PLLA microparticles of similar size or BSA alone or in another example with HA nanoparticles (approximately 250 nanometers, typical diameter), untagged HA nanoparticles of similar size or BSA alone. Because the same preparations and conditions were used in FIGS. 8A and 8B, FIG. 8B depicts data shown in FIG. 8A revealing that behavior of the particle preparations was similar. For example, none of the untagged particles (HA, NIPA, PLLA) or BSA alone (BSA) when injected into a host were able to significantly effect the amount of free and circulating BSA antibody detected in the serum. On the other hand, functionalizing a particle by tagging with BSA (HA-BSA, NIPA-BSA, PLLA-BSA) and introducing it into a host having circulating BSA antibodies resulted in the removal of the circulating BSA antibodies from the host's serum in less than a week.

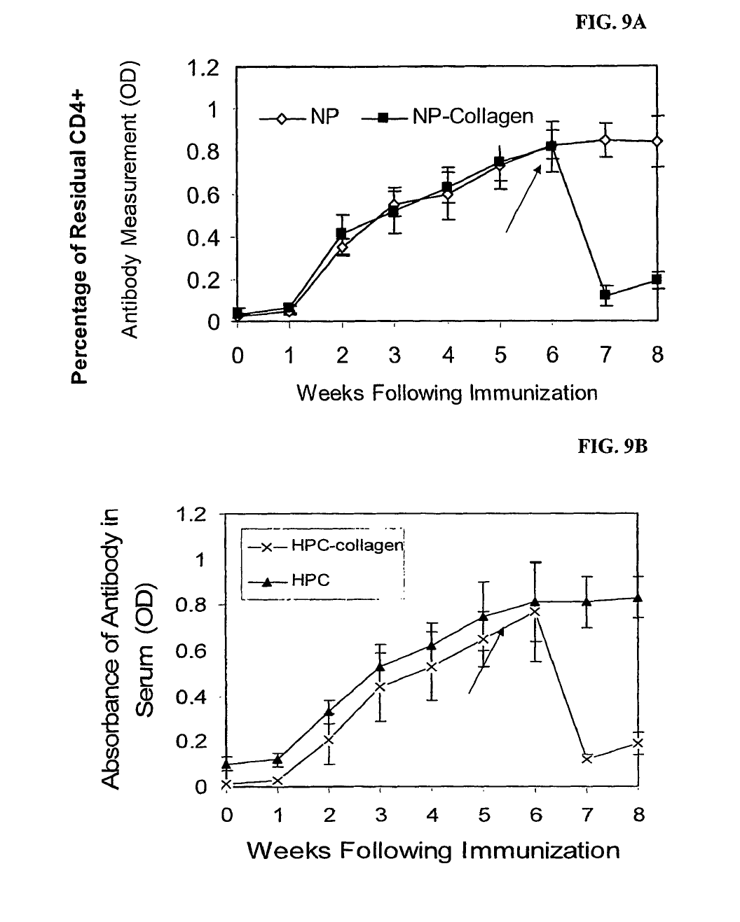

In still another example, Balb/C mice were immunized with bovine skin collagen following a standard immunization procedure as is known to one of ordinary skill in the art. At one week following immunization, mice began to produce antibodies against collagen. At the end of week 6 as depicted by the arrow in FIGS. 9A and 9B, mice were injected intravenously (via the tail vein) with particles tagged with and without bovine Type I collagen. Intravenous doses were about 2.0 mg particles/0.5 mL. FIGS. 9A and 9B are representative examples showing the in vivo efficacy of specifically tagged particles and their ability to remove a particular unwanted antibodies from circulation after allowing the unwanted antibody concentration to accumulate for six weeks. As shown in FIG. 9A, injection with untagged NIPA particles (approximately 100 nanometers, typical diameter; NP) did not affect collagen antibody concentration which continued to rise at week 7 and week 8. On the other hand, injection with tagged NIPA particles (conjugated with collagen; NP-Collagen) significantly reduced collagen antibody concentrations in the animals at week 7; the reduction was greater than 75% in just one week. Histologic analyses of tissue samples collected from some representative animals, including samples from the kidneys, lung, liver, brain, intestines, heart and spleen, were examined for particles. The analyses showed that NIPA particles were predominantly located in the liver (data not shown). This indicates that unwanted circulating antibodies in a host are recognized by a particle preparation specifically tagged to recognize the antibody and that following antibody recognition, such particle-antibody complexes are removed from the circulation and trapped in the liver where they are processed for elimination. Immunized mice not sacrificed were found to survive treatment with a particle preparation of the present invention and exhibited no physiologic or behavioral changes after treatment. These animals were also able to degrade the particle preparation (predominantly in the liver) and eliminate by-products of the preparation via hydrolysis (data not shown).

In FIG. 9B, the conditions as described for FIG. 9A were repeated using particle preparations tagged comprising HPC particles of 1-5 micrometers (typical diameter) conjugated with collagen (HPC-collagen) or untagged particles (HPC) injected into mice. FIG. 9B shows that immediately following injection with tagged HPC particles, as depicted by the arrow at week 6, the presence of the unwanted antibody was virtually absent in the serum as compared with no effect upon injection with untagged particles. Both tagged HPC particles and tagged NIPA particles exhibited very similar behavior at week 7 and week 8 as shown in FIGS. 9A and 9B indicating that functionalizing a particle as described herein with a specific recognition molecule to recognize the toxin promotes elimination of the unwanted toxin from the circulation.

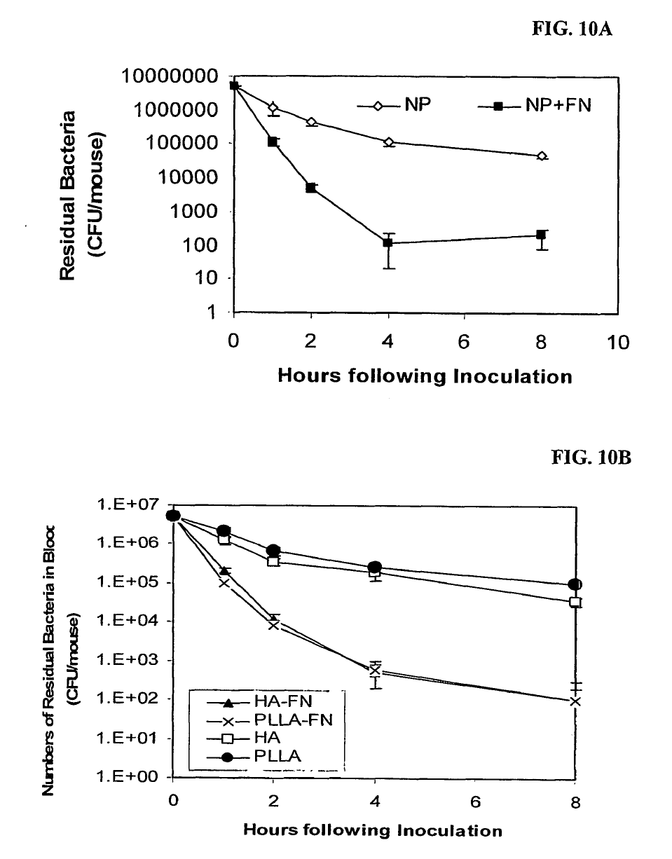

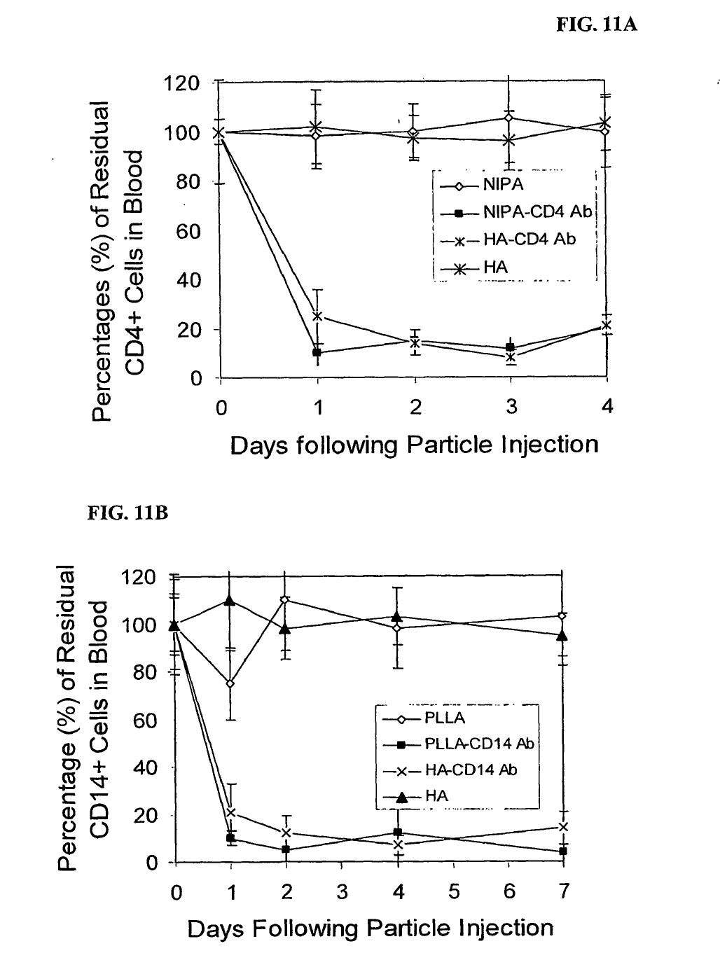

In yet another example, mice were infected with a toxic bacteria, Staphylococcus aureus, and the effects of particle injection were observed following its introduction. FIGS. 10A and 10B illustrate such effects and the ability of particle preparation of the present invention to reduce infection with a toxic bacteria by enhancing bacterial killing and reducing overall survival of the bacteria. FIGS. 10A and 10B also illustrate that functionalized particles when tagged with a specific recognition molecule that recognize the toxin (e.g., bacteria) are capable of recognizing the toxin, forming a complex with the toxin and removing it from the host's circulation. For FIGS. 10A and 10B, Balb/C mice were inoculated with a 1.2 mL solution containing S. aureus (approximately 5×10⁶ bacteria/mouse) by introduction into the peritoneal cavity. About ten minutes later, tagged particles or untagged particles were injected into the peritonea at about 1 mg particle dry weight/0.2 mL solution. In FIG. 10A, particles were NIPA nanoparticles (approximately 100 nanometers, typical diameter) untagged (NP) or tagged by conjugation with fibronectin (NP+FN), using fibronectin as a protein that specifically recognizes and binds fibronectin receptors expressed in high amounts on the cell surface of S. aureus. FIG. 10B shows data from two examples, one in which animals were injected with particles of PLLA (approximately 5 micrometers, typical diameter) untagged (PLLA) or tagged by conjugation with fibronectin (PLLA-FN) and another in which animals were injected with particles of HA of approximately 300 nanometers (typical diameter) untagged (HA) or tagged by conjugation with fibronectin (HA-FN). In each example, mice were sacrificed at specific times post-inoculation and the amount of free bacteria present in the peritonea was calculated using a pour plate technique.

FIG. 10A shows that following treatment of mice with functionalized NIPA particles (NP+FP), there was a virtual absence of free bacteria in the peritoneal cavity of the mouse as compared with only a small decline in bacterial count in animals treated with untagged NIPA particles (NP). FIG. 10B shows data from examples in which animals were injected with either functionalized and tagged HA particles as compared with untagged HA particles or functionalized and tagged PLLA particles as compared with untagged PLLA particles Like FIG. 10A, FIG. 10B shows that treatment of a host with functionalized and tagged particles significantly reduced bacterial count to a very negligible amount in the serum as compared with only slight reductions in bacterial count following treatment with untagged particles. The drastic reductions in circulating bacteria following introduction of the functionalized and tagged particles occurred less than four hours after-their introduction. Both FIG. 10A and FIG. 10B illustrate that introduction of particle preparations of the present invention to a host infected with a toxin resulted in rapid removal of the toxin from the host's circulation (akin to bacterial killing).

Referring now to FIGS. 11A and 11B, Balb/C mice were provided with an unwanted toxin by using an immunization strategy. Here, animals were immunized with bovine Type I collagen to boost the number of CD4+ cells and CD14+ cells in circulation. After four weeks, mice were injected intravenously and in separate examples with (a) functionalized particles tagged with CD4 antibodies or untagged particles or (b) functionalized particles tagged with CD14 antibodies or untagged particles. Mice were then sacrificed and blood samples from the heart was used to measure the amount of free CD4+ or CD14+ cells in circulation. FIGS. 11A and 11B are representative examples showing the effects of functionalized and tagged particles in a host provided with an unwanted toxin such as an artificial boosted immune system.

FIG. 11A shows combined data from separate examples in which animals were treated as described above and injected with (a) particles of NIPA (approximately 100 nanometers, typical diameter) untagged (NIPA) or tagged with CD4 antibodies (NIPA-CD4 Ab) or (b) particles of HA (approximately 300 nanometers, typical diameter) untagged (HA) or tagged with CD4 antibodies (HA-CD4 Ab). In FIG. 11A functionalized particles specifically tagged with a recognition molecule to recognize CD4+ cells (NIPA-CD4 Ab or HA-CD4 Ab) successfully depleted the unwanted CD4+ cells circulating in the blood by week one post-injection. On the other hand, circulating CD4+ cells remained in the circulation in a host treated with untagged particles (NIPA or HA). In fact, >90% of free CD4+ cells were removed from circulation within one week following injection with functionalized and tagged NIPA particles and about 80% of free CD4+ cells were removed from circulation within one week following treatment with functionalized and tagged HA particles.

FIG. 11B shows combined data from separate examples in which animals were treated as described above and injected with (a) particles of PLLA (approximately 5 micrometers, typical diameter) untagged (PLLA) or tagged with CD14 antibodies (PLLA-CD14 Ab) or (b) particles of HA (approximately 150 nanometer, typical diameter) untagged (HA) or tagged with CD14 antibodies (HA-CD14 Ab). In FIG. 11B, functionalized particles specifically tagged with a recognition molecule to recognize CD14 cells (PLLA-CD14 Ab or HA-CD14 Ab) successfully depleted unwanted CD14+ cells circulating in the blood by week one post-injection while CD14+ cells remained unchanged in a host treated with untagged particles (PLLA or HA). Both FIGS. 11A and 11B show that unwanted CD+ cells may be removed by particles specifically functionalized and tagged to recognize a portion of the cell. Removal relies on the tag (specific recognition molecule) provided to the particle that is used; the particle behaving similar to a carrier. When combined, the tagged particle is capable of recognizing the unwanted toxin and removing it from circulation and eliminate it from the body.

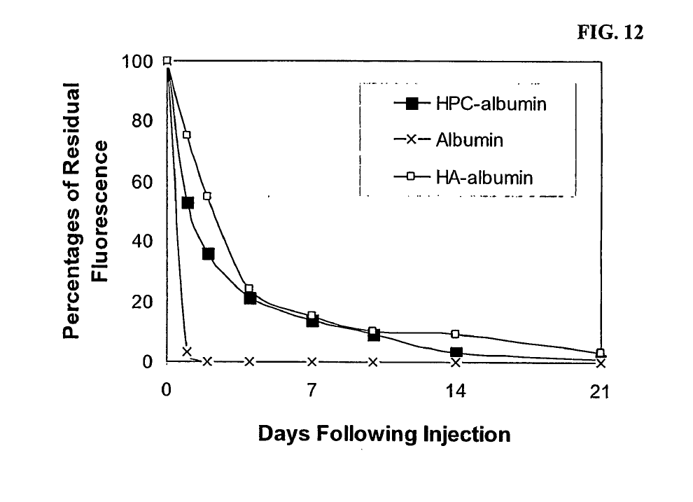

Observations of functionalized and tagged particles of the present invention and their behavior in a host show that, when functionalized and tagged, the particle preparations are capable of remaining in circulation for several weeks. Accordingly, by conjugating a toxin-specific recognition molecule or ligand-masking moieties (e.g., protein, peptide, antibody, or other molecular species) to a functionalized particle of the present invention, the half life of the toxin-specific recognition molecule or ligand-masking moieties will dramatically increase. For example, molecules with a half life of 2-4 hours will persist in the circulation of more than 7 days and as long as three weeks when tagged to a functionalized particle of the present invention, as shown in FIGS. 5A and 12.

In FIG. 12, mice were injected with functionalized and tagged particles or just a functionalized tag. Tags were provided as described in FIGS. 5A and 5B, labeled with FITC, and viewed by fluorometry in serum samples taken at specific time points. The tags includes HPC particles of approximately 10 micrometers (typical diameter) provided untagged (HPC) or tagged by conjugating to albumin (HPC-albumin) or HA particles of approximately 300 nanometers (typical diameter) provided untagged (HA) or tagged by conjugating to albumin (HA-albumin). FIGS. 5A and 12 illustrate that particles of the present invention functionalized and tagged with a toxin-specific recognition molecule or counter ligand are more effective, last longer in a host's circulation and, as such, are better suited for use in a host in need thereof.

Particles of the present invention may also be incorporated with an activator or inhibitor to one or more specific toxic agents, thereby useful in neutralizing the toxic agent. Particles as described herein may be used in vitro, ex vivo, or in vivo to achieve their effects.

Additional objects, advantages and novel features of the invention as set forth in the description, will be apparent to one skilled in the art after reading the foregoing detailed description or may be learned by practice of the invention. The objects and advantages of the invention may be realized and attained by means of the instruments and combinations particularly pointed out here.

What is claimed is:

1. A particle preparation for recognizing a toxin in a host comprising:
a particle with a typical diameter of less than 100 micrometers having a tag in direct contact with a functionalized portion of the particle, the particle comprising a polymer and the functionalized portion selected from the group consisting of amine-reactive group, carboxyl-reactive group, hydroxyl-reactive group, sulfonic-reactive group, and aldehyde-reactive group,
wherein the tag is specifically recognized by the toxin, and
wherein the one or more particles are introduced into the host having a toxin, recognize the toxin and the toxin is reduced in the host.

2. The particle preparation of claim 1, wherein the one or more particles further comprise a coating selected from the group consisting of a surfactant, light-emitting species, radioactive species, nuclear species, and combinations thereof.

3. The particle preparation of claim 1, wherein the polymer is selected from the group consisting of polyelectrolyte, hydroxypropyl cellulose, N-isopropylacrylamide, hyaluronan, poly-L-lactic acid, polysaccharide, poly-L-glycolic acid, protein, peptide, and combinations thereof.

4. The particle preparation of claim 1, wherein the toxin is selected from the group consisting of antibody, immune product, host cell by-product or metabolite, microorganism, product produced from a microorganism, and combinations thereof.

5. The particle preparation of claim 1, wherein the one or more particles remove the toxin from the host.

6. The particle preparation of claim 1, wherein the one or more particles are introduced into the host by one of the group consisting of injection, infusion, inhalation, by mouth, by drops, transdermally, and by suppository.

7. The particle preparation of claim 6 further comprising a pharmaceutically acceptable carrier.

8. A method for recognizing a toxin in a host comprising:
introducing one or more particles with a typical diameter of less than 100 micrometers into a host having toxins, the one or more particles each having a tag in direct contact with a functionalized portion of the particle, the one or more particles comprising a polymer, the functionalized portion selected from the group consisting of amine-reactive group, carboxyl-reactive group, hydroxyl-reactive group, sulfonic-reactive group, and aldehyde-reactive group, and the tag is specifically recognized by the toxin,
wherein the one or more particles recognize the toxin and the toxin is reduced in the host.

9. The method of claim 8, wherein the one or more particles further comprise a coating selected from the group consisting of a surfactant, light-emitting species, radioactive species, nuclear species, and combinations thereof.

10. The method of claim 8, wherein the polymer is selected from the group consisting of polyelectrolyte, hydroxypropyl cellulose, N-isopropylacrylamide, hyaluronan, poly-L-lactic acid, polysaccharide, poly-L-glycolic acid, protein, peptide, and combinations thereof.

11. The method of claim 8, wherein the toxin is selected from the group consisting of antibody, immune product, host cell by-product or metabolite, microorganism, product produced from a microorganism, and combinations thereof.

12. The method of claim 8, wherein the one or more particles remove toxins from the host.

13. The method of claim 8, wherein the one or more particles are introduced into the host by one of the group consisting of injection, infusion, inhalation, by mouth, transdermally, and by suppository.

14. The method of claim 13, wherein the one or more particles further comprise a pharmaceutically acceptable carrier.

15. The method of claim 8, wherein the tag includes a drug, antibody, ligand, antigen, protein, peptide, counter-ligand moiety, fatty acid moiety, and carbohydrate moiety.

16. A particle preparation for recognizing a toxin in a host comprising:
a particle with a typical diameter of less than 100 micrometers having a tag in direct contact with a functionalized portion of the particle, the particle comprising a polymer and the functionalized portion selected from the group consisting of amine-reactive group, carboxyl-reactive group, hydroxyl-reactive group, sulfonic-reactive group, and aldehyde-reactive group,
wherein the tag is specifically recognized by the toxin, and
wherein the one or more particles are introduced into the host having a toxin, recognize the toxin and the toxin is removed from the host.

17. The particle preparation of claim 16, wherein the polymer is selected from the group consisting of polyelectrolyte, hydroxypropyl cellulose, N-isopropylacrylamide, hyaluronan, and poly-L-lactic acid.

18. The particle preparation of claim 16, wherein toxin is selected from the group consisting of antibody and molecule displayed on the toxin surface.

19. The particle preparation of claim 16, wherein the tag includes fibronectin, collagen, CD+ cell antibody, BSA, and albumin.

20. The particle preparation of claim 16, wherein the particle is introduced by injection.
